(19) United States
(12) Patent Application Publication
(10) Pub. No.: US 2023/0404094 A1
(43) Pub. Date: Dec. 21, 2023

(54) FERMENTED MILK FOR REGULATING GUT HEALTH AND PREPARATION METHOD THEREOF

(71) Applicant: Beijing Sanyuan Foods Co., Ltd., Beijing (CN)

(72) Inventors: Lijun CHEN, Beijing (CN); Tiemin Jiang, Beijing (CN); Xiaohong Song, Beijing (CN); Jiantao Li, Beijing (CN); Junying Zhao, Beijing (CN); Bin Liu, Beijing (CN); Weicang Qiao, Beijing (CN); Yanpin Liu, Beijing (CN)

(21) Appl. No.: 18/335,657

(22) Filed: Jun. 15, 2023

Related U.S. Application Data
(63) Continuation of application No. PCT/CN2023/073821, filed on Jan. 30, 2023.

(30) Foreign Application Priority Data
Apr. 22, 2022 (CN) 202210427756.9

(51) Int. Cl. A23C 9/127; A23C 9/13; A61K 35/747; A61K 35/20; A61K 38/47

(52) U.S. Cl. CPC A23C 9/1275; A23C 9/1307; A61K 35/747; A61K 35/20; A61K 38/47; C12Y 302/01108; A23V 2400/249; A23V 2400/169; A23V 2400/113

(57) ABSTRACT

Raw materials of a fermented milk include partially degreased raw milk, 4-10% w/w of saccharide based on the mass of the raw milk, 0.06-0.15% w/w of lactase based on the mass of the raw milk, 0.03-0.18% w/w of a stabilizer based on the mass of the raw milk, and 20-50 dcu of fermenting bacteria added per 100 kg of the raw milk; and the fermenting bacteria is one or more selected from the group consisting of Lactobacillus paracasei, Lactobacillus plantarum, Lactobacillus acidophilus, Bifidobacterium lactis and Streptococcus thermophilus, and preferably one or more selected from the group consisting of Lactobacillus plantarum with a preservation number of CGMCC No. 19748, Lactobacillus acidophilus with a preservation number of CGMCC No. 1084 and Streptococcus thermophilus. According to the fermented milk and a preparation method thereof, the prepared fermented milk has a more stable state during the shelf life.

### CROSS REFERENCE

The present application claims priority to Chinese patent application No. 202210427756.9, filed on Apr. 22, 2022 at the Chinese Patent Office, and entitled “Fermented Milk for Regulating Gut Health and Preparation Method Thereof”, the entire contents of which are incorporated herein by reference.

### TECHNICAL FIELD

The present application relates to the field of fermented dairy products, in particular to a fermented milk and a preparation method thereof.

### BACKGROUND ART

There is growing evidence that human gut microbiota (GM) may be a useful marker and contributor to the diagnosis, treatment and prevention of many human diseases, such as obesity, diabetes, liver diseases, cancers and neurodegenerative diseases. Gut microbiota is a complex microecosystem that remains relatively stable throughout the life cycle, but fluctuates every day and is significantly affected by dietary changes. Fermented foods, especially fermented milk, contain viable bacteria, such as bifidobacteria, lactobacilli and streptococci, which are some of the major sources of transient bacteria in the human gut tract and may affect the structure and function of gut microbiota. Many previous studies have shown that consumption of fermented milk has a positive effect on gut microbiota composition and host health, including promoting the reproduction of probiotics: lactobacillus and bifidobacterium. However, other studies have shown no or opposite effects. This difference may be mainly due to differences in nutrition and chemical composition of the food, strains and research duration.

Palmitoleic acid (C16:1 n-7), a hexadecenoic acid, has been identified as a regulator of physiological myocardial dystrophy and may have a protective effect on myocardial fibrosis and inflammation. There is a significant negative correlation between the C16:1 n-7 level in human body and the incidence rate of type 2 diabetes. In vitro studies suggest that C16:1 n-7 may advantageously target the pancreas and modulate B-cell function. However, foods rich in C16:1 n-7 are currently lacking. Gut microbiota is closely related to human health. Consumption of fermented milk has a positive impact on the composition of gut microbiota and host health, while different fermentation raw materials, raw material ratios and fermentation processes affect fermentation metabolites, thereby producing different health effects. However, there is no new fermented milk created from the production of healthy fermentation metabolites in the existing products.

In view of this, the present application is proposed.

### SUMMARY OF THE INVENTION

The purpose of the present application is to provide a fermented milk for regulating gut health and a preparation method thereof. In the preparation method, lactase is added to hydrolyze lactose, which reduces the added amount of additional saccharide in the raw materials, at the same time shortens the time required for reaction, further reduces the caloric content of the product, retains the nutrition of the product, and produces fermentation metabolites conducive to health.

In order to achieve the above purpose, it is characterized in that, Raw materials of the fermented milk comprise: partially degreased raw milk, 4-10% w/w of saccharide based on the mass of the raw milk, 0.06-0.15% w/w of lactase based on the mass of the raw milk, 0.03-0.18% w/w of a stabilizer based on the mass of the raw milk, and 20-50 dcu of fermenting bacteria added per 100 kg of the raw milk;

And the fermenting bacteria is one or more selected from the group consisting of Lactobacillus paracasei, Lactobacillus plantarum, Lactobacillus acidophilus, Bifidobacterium lactis and Streptococcus thermophilus, and preferably one or more selected from the group consisting of Lactobacillus plantarum with a preservation number of CGMCC No. 19748, Lactobacillus acidophilus with a preservation number of CGMCC No. 1084 and Streptococcus thermophilus.

Preferably or alternatively, the saccharide is one or more selected from the group consisting of white granulated sugar, glucose-fructose syrup and fructose.

Preferably or alternatively, the stabilizer is one or more selected from the group consisting of pectin, gellan gum, starch, soluble soy polysaccharide, sodium carboxymethyl cellulose, agar, and gelatin, and preferably one or more selected from the group consisting of pectin, agar, starch, and gelatin, added in an amount of 0.04-0.16% based on the mass of the raw milk.

Preferably or alternatively, the fermented milk has a C16:1n7 content of not less than 1.12μg/mg.

On the other hand, the present application also provides a preparation method of the above fermented milk, which is performed with the following steps in sequence:
(1) Preparing a mixed emulsion with the raw milk, saccharide, stabilizer and lactase;
(2) Inoculating the mixed emulsion prepared in step (1) with fermenting bacteria, and performing fermentation under heat preservation; and
(3) Performing demulsification, chilling, filling, refrigeration and post-ripening to obtain the fermented milk;
Wherein the method for preparing the mixed emulsion in step (1) comprises: mixing the raw milk with the saccharide, stabilizer and lactase, performing hydrolyzation under heat preservation, homogenization, heating and cooking, and cooling to obtain the mixed emulsion; or, mixing the raw milk with 75-85% of the saccharide and the lactase at first, performing hydrolyzation under heat preservation, heating and cooking, cooling, adding the remaining saccharide and the stabilizer, and performing homogenization and sterilization to obtain the mixed emulsion.

Preferably or alternatively, the time for hydrolyzation under heat preservation in step (1) is 40 to 80 min.

Preferably or alternatively, the pressure for homogenization in step (2) is 150 to 200 bar.

Preferably or alternatively, the time for heat preservation in step (2) is 90 to 150 min.

Preferably or alternatively, the time for hydrolyzation under heat preservation in step (3) is 5 to 6 h.

According to the fermented milk and the preparation method thereof provided by the present application, through improvement of fermentation strains in combination with improvement of production process, the prepared fermented milk has a higher number of active lactic acid bacteria and a more stable state during the shelf life, contains a variety of unique substances beneficial to human health, and can effectively improve the number and state of beneficial microbial populations in human gut tract.

### BRIEF DESCRIPTION OF THE DRAWINGS

FIG. 1 shows a fatty acid volcano plot of the fermented milk product prepared in Example 1 and a conventional fermented milk product;

FIG. 2 shows PERMANOVA analysis of β diversity based on weighted Unifrac distance for the two groups;

FIG. 3 shows NMDS analysis of β diversity based on weighted Unifrac distance for the two groups;

FIG. 4 shows a bar graph of LEfSe of gut microbiota in the fermented milk group and conventional fermented milk group; and

FIG. 5 shows a phylogenetic tree and heat map of gut microbiota distribution in the fermented milk group and conventional fermented milk group.

### DETAILED DESCRIPTION OF THE INVENTION

The specific embodiments of the present application are described in detail below. It should be understood that the specific embodiments described herein are only used to illustrate and explain the present application and are not intended to limit the present application.

The experimental methods used in the following examples are conventional methods unless otherwise specified.

The materials, reagents, etc. used in the following examples can be obtained commercially without special instructions.

### Example 1

This example provides a fermented milk product.

The fermented milk product was prepared according to the following process:
Raw milk was pasteurized, and cooled to room temperature after sterilization;
The raw milk was preheated to 30° C., 4% of white granulated sugar, 0.12% of lactase and 0.12% of agar, based on the mass of the raw milk were added to the raw milk, and the feed liquid was hydrolyzed at 30° C. for 50 min under heat preservation;
The feed liquid was preheated to 70° C. after hydrolyzation, homogenized under a pressure of 200 bar, sterilized at 90-95° C. for 300 s after homogenization, and heated and cooked at 100° C. for 120 min after sterilization;
The cooked feed liquid was cooled to 43±2° C., inoculated with a compound strain of Lactobacillus thermophilus, Lactobacillus plantarum (preservation number: CGMCC No. 19748) and Lactobacillus acidophilus(preservation number: CGMCC No. 1084) with a ratio of (8:1:1) in an amount of 50 dcu per 100 kg of the raw milk, fermented for 6 h under heat preservation after inoculation, demulsified after fermentation, chilled, filled into a package, cooled to 2-8° C., and post-ripened to obtain the fermented milk product.

The fermented milk product prepared in this example is a drinkable fermented milk product.

### Example 2

This example provides a fermented milk product.

The fermented milk product was prepared according to the following process:
Raw milk was pasteurized, and cooled to room temperature after sterilization;
The raw milk was preheated to 40° C., 5.5% of white granulated sugar and 0.08% of lactase, based on the mass of the raw milk, were added to the raw milk, and the feed liquid was hydrolyzed at 40° C. for 80 min under heat preservation;
The feed liquid was heated and cooked at 100° C. for 2.5 h after hydrolyzation, cooled to room temperature, 1.5% of white granulated sugar and 0.06% of gelatin based on the mass of the raw milk were added thereto, the feed liquid was homogenized under a pressure of 150 bar, and sterilized at 90-95° C. for 300 s after homogenization;
The sterilized feed liquid was cooled to 43±2° C., inoculated with Lactobacillus acidophilus (preservation number: CGMCC No. 1084) in an amount of 20 dcu per 100 kg of the raw milk, fermented for 5 h under heat preservation, demulsified after fermentation, chilled, filled into a package, cooled to 2-8° C., and post-ripened to obtain the fermented milk product.

The fermented milk product prepared in this example is a stirred fermented milk product.

### Example 3

This example provides a fermented milk product.

The fermented milk product was prepared according to the following process:
Raw milk was pasteurized, and cooled to room temperature after sterilization;
The raw milk was preheated to 50° C., 10% of white granulated sugar, 0.15% of lactase and 0.12% of pectin, based on the mass of the raw milk were added to the raw milk, and the feed liquid was hydrolyzed at 50° C. for 40 min under heat preservation;
The feed liquid was preheated to 70° C. after hydrolyzation, homogenized under a pressure of 200 bar, sterilized at 90-95° C. for 300 s after homogenization, and heated and cooked at 100° C. for 90 min after sterilization;
The cooked feed liquid was cooled to 43±2° C., inoculated with Lactobacillus plantarum (preservation number: CGMCC No. 19748) in an amount of 35 dcu per 100 kg of the raw milk, fermented for 5.5 h under heat preservation, demulsified after fermentation, chilled, filled into a package, cooled to 2-8° C., and post-ripened to obtain the fermented milk product.

The fermented milk product prepared in this example is a drinkable fermented milk product.

### Effect Example 1

The viscosity changes of the fermented milks prepared in Example 1 and Example 2 during the shelf life were determined. The results are shown in Table 1, indicating that the prepared drinkable yogurt and stirred yogurt have different viscosity characteristics.

TABLE 1
Viscosity changes of fermented milk products
of the examples during shelf life

Viscosity

| Storage time | Example 1 | Example 2 |
|---|---|---|
| Semi-finished product | 856 | 5738 |

| | | | |
|---|---|---|---|
| 1 | day | 972 | 7758 |
| 5 | days | 1530 | 8438 |
| 12 | days | 1793 | 9717 |
| 18 | days | 2034 | 11757 |
| 21 | days | 2150 | 11830 |

### Effect Example 2

Known primary metabolites in the fermented milk prepared in Example 1 and commercially available conventional fermented milk were determined by nuclear magnetic resonance. The results are shown in Table 2.

TABLE 2
Differential metabolites in products determined by nuclear magnetic resonance

| | | | | Normal | |
|---|---|---|---|---|---|
| | Molecular | | Example | fermented | P (FDR- |
| Name | formula | Classification | 1 (mM) | milk (mM) | corrected) |
| Hydroxyacetone | C₃H₆O₂ | Alcohol | 0.020 ± 0.001 | 0.000 ± 0.000 | 3.83E−05 |
| (HA) | | | | | |
| Creatinine | C₄H₇N₃O | Amino acid | 0.042 ± 0.008 | 0.018 ± 0.001 | 0.021 |
| | | derivative | | | |
| Creatine | C₄H₉N₃O₂ | Amino acid | 0.039 ± 0.006 | 0.067 ± 0.006 | 0.015 |
| | | derivative | | | |
| Alanine | C₃H₇NO₂ | Amino acid | 0.02 ± 0.001 | 0.015 ± 0.001 | 0.024 |
| Betaine | C₅H₁₁NO₂ | Amino acid | 0.019 ± 0.003 | 0.007 ± 0.001 | 0.006 |
| Lactose | C₁₂H₂₂O₁₁ | Disaccharide | 5.876 ± 1.432 | 12.893 ± 0.547 | 0.006 |
| Sucrose | C₁₂H₂₂O₁₁ | Disaccharide | 19.288 ± 0.842 | 22.867 ± 0.925 | 0.021 |
| Acetoin | C₄H₈O₂ | Ketone | 0.090 ± 0.012 | 0.065 ± 0.003 | 0.050 |
| Dihydroxyacetone | C₃H₆O₃ | Monosaccharide | 0.015 ± 0.001 | 0.007 ± 0.000 | 0.004 |
| DHA | C₆H₁₂O₆ | Monosaccharide | 6.255 ± 0.132 | 0.134 ± 0.021 | 4.96E−06 |
| Fructose | C₆H₁₂O₆ | Monosaccharide | 6.007 ± 0.129 | 2.23 ± 0.180 | 6.41E−05 |
| Galactose | C₆H₁₂O₆ | Monosaccharide | 13.618 ± 0.385 | 0.365 ± 0.081 | 8.54E−06 |
| Glucose | C₆H₁₂O₆ | Monosaccharide | 0.068 ± 0.007 | 0.000 ± 0.000 | 0.001 |
| Mannose | C₅H₈O₃ | Organic acid | 0.005 ± 0.000 | 0.006 ± 0.001 | 0.021 |
| 2-oxovalerate | C₃H₄O₃ | Organic acid | 0.044 ± 0.004 | 0.025 ± 0.008 | 0.050 |

As can be seen from Table 2, the lactose content of the fermented milk product of Example 1 is significantly reduced, while the content of DHA is significantly higher than that of the conventional fermented milk product. It can be seen that the fermented milk product provided by the present application has significantly reduced the lactose content and increased consumer acceptance through optimization of fermentation process and fermentation strains; and improves the content of functional substance DHA in fermentation products, which is helpful to improve consumer health.

### Effect Example 3

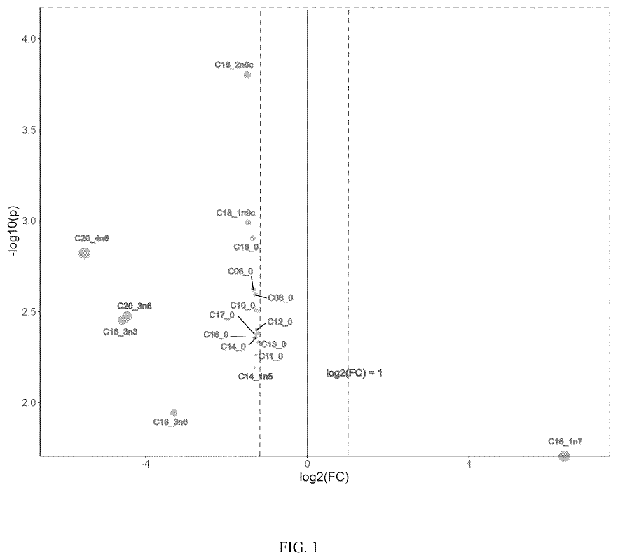

The fatty acid profiles of the fermented milk product prepared in Example 1 and a commercially available conventional fermented milk product were determined using gas chromatography. The results are shown in FIG. 1 and Table 3.

The novel lipid factor C16:1n7 was only detected in the fermented milk product prepared in Example 1. C16:1n7 is an ω-7 polyunsaturated fatty acid known to have multiple effects in the host including the prevention or amelioration of inflammation, obesity, diabetes and other chronic metabolic diseases.

The above results demonstrate that the fermented milk products provided herein can produce a variety of unique functional fermentation products through optimization of fermentation process and fermentation strains. In particular, the content of C16:1n7 in the product can reach 1.12 μg/mg, which is not detected in the conventional fermented milk commercially available for comparison.

TABLE 3
Comparison of fatty acid composition of example
product and conventional fermented product

| | | Conventional | |
|---|---|---|---|
| Name | Example 1 | fermented milk | p# |
| C6:0 | 1.37 ± 0.36 | 3.46 ± 0.21 | ** |
| C8:0 | 0.84 ± 0.23 | 2.05 ± 0.16 | ** |
| C10:0 | 1.87 ± 0.52 | 4.51 ± 0.34 | ** |
| C11:0 | 0.04 ± 0.01 | 0.10 ± 0.01 | ** |
| C12:0 | 2.14 ± 0.61 | 5.11 ± 0.36 | ** |
| C13:0 | 0.07 ± 0.02 | 0.17 ± 0.01 | ** |
| C14:0 | 6.94 ± 1.96 | 16.82 ± 1.02 | ** |
| C14:1n5 | 0.57 ± 0.16 | 1.41 ± 0.05 | ** |
| C16:0 | 23.53 ± 6.50 | 57.62 ± 3.13 | ** |
| C16:1n7 | 1.12 ± 0.27 | NA | ** |
| C17:0 | 0.42 ± 0.11 | 1.00 ± 0.05 | ** |
| C18:0 | 7.35 ± 1.68 | 18.75 ± 1.09 | ** |
| C18:1n9c | 15.23 ± 3.40 | 42.01 ± 1.89 | ** |
| C18:2n6c | 1.90 ± 0.25 | 5.34 ± 0.31 | *** |

### Effect Example 4

Efficacy evaluation experiments were performed on the fermented milk product prepared in Example 1 and a commercially available conventional fermented milk product.

A total of 95 healthy adults (38 males and 57 females) were enrolled in the study, ranging in age from 22 to 58 years, and written informed consent was obtained from both parties. None of the participants had known metabolic or gastrointestinal disorders or had received antibiotics within 3 months prior to study initiation. During the experiment, all participants were allowed to maintain their normal lifestyle and diet, but were asked to avoid consuming any other fermented milk, probiotic or antibiotic preparations, and were asked to record a 72-hour diet diary before collecting stool samples.

According to gender and age, participants were randomly assigned to the example group (n=48; 19 males; average age 36.1±8.6 years) or the conventional fermented milk group (n=47; 19 males; average age 35.6±9.4 years). From day 0, all participants were asked to drink a cup of 100 ml fermented milk in the morning and in the evening, with the example group drinking the fermented milk product prepared in Example 1 and the conventional fermented milk group drinking a commercially available conventional fermented milk product, for 28 days, followed by a 10-day follow-up period without consumption of fermented milk, during which each participant was still asked not to consume a product containing probiotics or prebiotics. Each participant provided stool samples on days 0, 7, 14, 21, 28, and 38 and stored at −80° C. for subsequent analysis.

A total of 415 stool samples were collected from 70 participants.

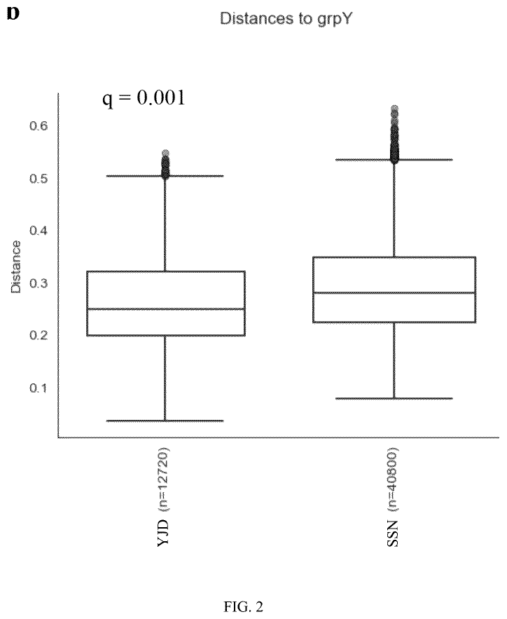

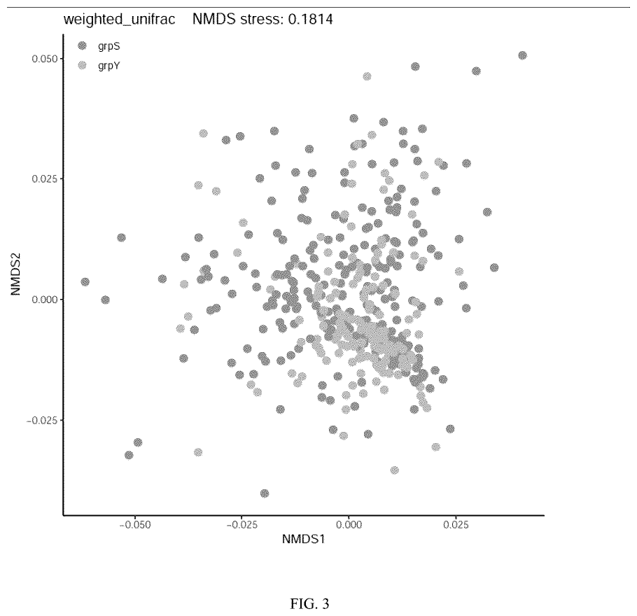

Each stool sample was sequenced to evaluate β diversity of each sample. The results are shown in FIGS. 2-3.

As can be seen from FIGS. 2 and 3, there is a significant difference in bacterial colony structure between the example group and the conventional fermented milk group in β diversity of gut microbiota.

There are significant changes in at least one intervention period for 21 and 18 genera in the example group and the conventional fermented milk group, respectively. For the example group, the abundance of Akkermansia muciniphila peaked at 21 days of consumption of the fermented milk, the abundance at day 21 was significantly higher than those at day 0, day 28 or day 38, and consistent changes were found at different classification levels of the analysis. However, there was no significant change in the abundance of Ruminococcus or Akkermansia muciniphila in the conventional fermented milk group. As the only member of Verrucomicrobia, Akkermansia muciniphila is considered to be the next generation of beneficial microorganisms, and has been proved to produce butyric acid, which is low under certain pathological conditions, such as obesity, T2DM, hypertension and liver diseases. Compared with baseline and follow-up period, its abundance increased significantly after ingestion of fermented milk.

At the same time, in the example group, the abundances of other beneficial microorganisms, such as Rumococcus, Anaerobes, Veronella and Bifidobacterium, also increased significantly, and the abundances of potentially harmful Staphylococcus aureus and Haemophilus parainfluenzae decreased.

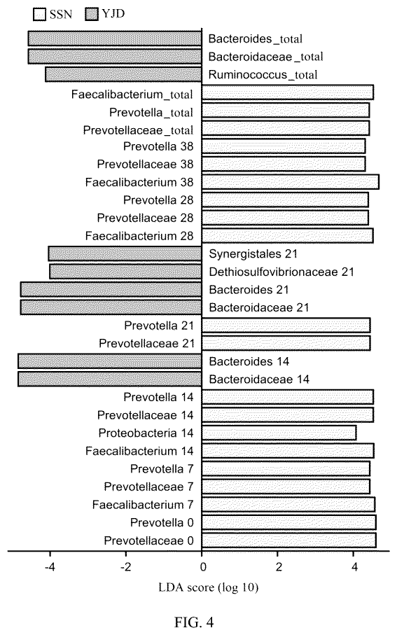

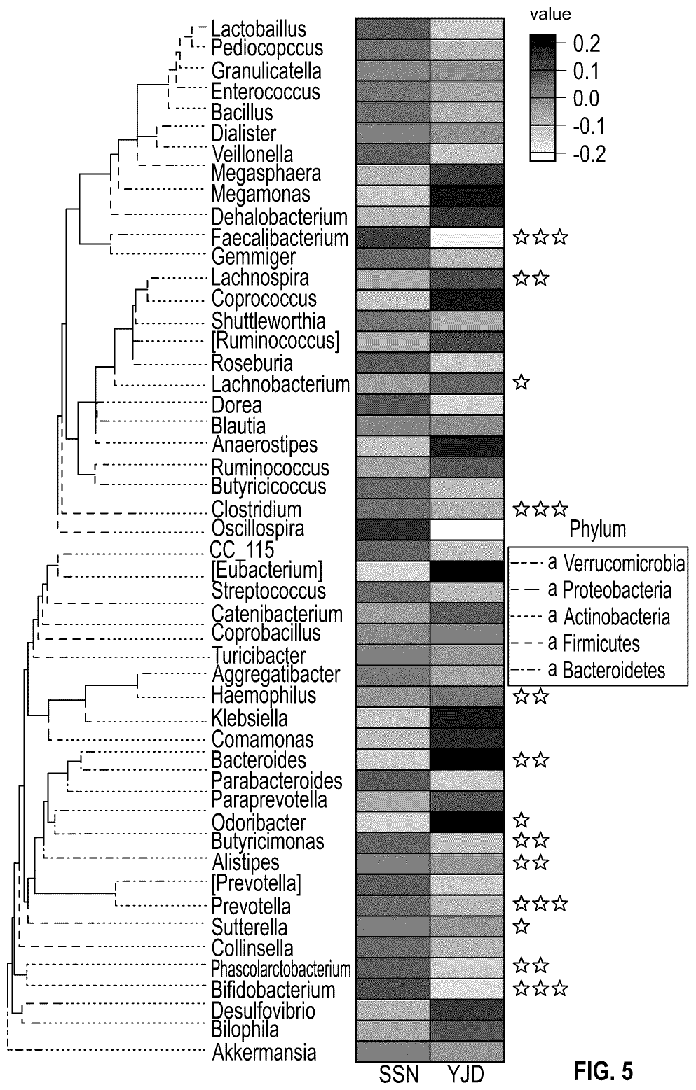

There were no significant differences in the abundance of any taxa between groups at baseline. However, as shown in FIGS. 4-5, significant differences in gut microbial composition began to develop between the two groups after 7 days. In the example group (SSN), Faecalibacterium, Prevotella and Prevotaceae are more abundant, while in the conventional fermented milk group (YJD), Bacaeroides, Ruminants and Bacteroidaceae are more abundant.

Correlation analysis showed that C16:1n7 (1.12±0.27 μg/mg) had a strong positive correlation with Faecalibacterium (p<0.001) and Bifidobacterium longum (p=0.01), and had a negative correlation with Coprococcus, Eubacterium, Bacteroides and Ruminococcus (p≤0.01), fully indicating that the fermented milk prepared in the present application is rich in functional components that can positively improve gut microorganisms.

The above differences suggest that consumption of different types of fermented milk may have an effect on gut microbial structure of healthy adults, while consumption of the fermented milk product prepared in the present application is more conducive to the growth of beneficial gut microorganisms than conventional fermented milk products.

To sum up, according to the present application, through improvement of fermentation strains in combination with improvement of production process, the prepared fermented milk product has a higher number of active lactic acid bacteria and a more stable state during the shelf life, contains a variety of unique substances beneficial to human health, and can effectively improve the number and state of beneficial microbial populations in human gut tract.

Although the preferred embodiments of the present application have been described in detail above, the present application is not limited to the specific details of the above embodiments, and various simple modifications can be made to the technical solutions of the present application within the scope of the technical concept of the present application, which fall within the protection scope of the present application. In addition, any combination of the various embodiments of the present application can be made without departing from the spirit of the present application, which should also be considered disclosed herein.

### Industrial Applicability

The present application relates to a fermented milk for regulating gut health. The raw materials of the fermented milk comprise: partially degreased raw milk, 4-10% w/w of saccharide based on the mass of the raw milk, 0.06-0.15% w/w of lactase based on the mass of the raw milk, 0.03-0.18% w/w of a stabilizer based on the mass of the raw milk, and 20-50 dcu of fermenting bacteria added per 100 kg of the raw milk. According to the present application, through improvement of fermentation strains in combination with improvement of production process, the prepared fermented milk product has a higher number of active lactic acid bacteria and a more stable state during the shelf life, contains a variety of unique substances beneficial to human health, and can effectively improve the number and state of beneficial microbial populations in human gut tract.

1-9. (canceled)

10. A fermented milk for regulating gut health, wherein raw materials of the fermented milk comprise: partially degreased raw milk, 4-10% w/w of saccharide based on the mass of the raw milk, 0.06-0.15% w/w of lactase based on the mass of the raw milk, 0.03-0.18% w/w of a stabilizer based on the mass of the raw milk, and 20-50 dcu of fermenting bacteria added per 100 kg of the raw milk;
And the fermenting bacteria is one or more selected from the group consisting of Lactobacillus paracasei, Lactobacillus plantarum, Lactobacillus acidophilus, Bifidobacterium lactis and Streptococcus thermophilus.

11. The fermented milk according to claim 10, wherein the fermenting bacteria is one or more selected from the group consisting of Lactobacillus plantarum with a preservation number of CGMCC No. 19748, Lactobacillus acidophilus with a preservation number of CGMCC No. 1084 and Streptococcus thermophilus.

12. The fermented milk according to claim 10, wherein the saccharide is one or more selected from the group consisting of white granulated sugar, glucose-fructose syrup and fructose.

13. The fermented milk according to claim 10, wherein the stabilizer is one or more selected from the group consisting of pectin, gellan gum, starch, soluble soy polysaccharide, sodium carboxymethyl cellulose, agar, and gelatin, added in an amount of 0.04-0.16% based on the mass of the raw milk.

14. The fermented milk according to claim 13, wherein the stabilizer is one or more selected from the group consisting of pectin, agar, starch, and gelatin. (New) The fermented milk according to claim 10, wherein the fermented milk has a C16:1n7 content of not less than 1.12 μg/mg.

16. A preparation method of the fermented milk according to claim 10, performed with the following steps in sequence:
(1) Preparing a mixed emulsion with the raw milk, saccharide, stabilizer and lactase;
(2) Inoculating the mixed emulsion prepared in step (1) with fermenting bacteria, and performing fermentation under heat preservation; and
(3) Performing demulsification, chilling, filling, refrigeration and post-ripening to obtain the fermented milk;
Wherein the method for preparing the mixed emulsion in step (1) comprises: mixing the raw milk with the saccharide, stabilizer and lactase, performing hydrolyzation under heat preservation, homogenization, heating and cooking, and cooling to obtain the mixed emulsion; or, mixing the raw milk with 75-85% of the saccharide and the lactase at first, performing hydrolyzation under heat preservation, heating and cooking, cooling, adding the remaining saccharide and the stabilizer, and performing homogenization and sterilization to obtain the mixed emulsion.

17. The preparation method of the fermented milk according to claim 16, wherein the time for hydrolyzation under heat preservation in step (1) is 50 to 80 min.

18. The preparation method of the fermented milk according to claim 17, wherein the pressure for homogenization in step (2) is 150 to 200 bar.

19. The preparation method of the fermented milk according to claim 18, wherein the time for heating and cooking is 1.5 to 3.5 h.

20. The preparation method of the fermented milk according to claim 19, wherein the time for hydrolyzation under heat preservation in step (3) is 5 to 6 h.
